(19) United States
(12) Plant Patent Application Publication
(10) Pub. No.: US 2012/0246787 P1
(43) Pub. Date: Sep. 27, 2012

(54) Calibrachoa plant named 'KLECA10217'

(50) ;

(75) Inventors: Nils Klemm, Stuttgart (DE); Anita Stöver, Ostfildern (DE)

(73) Assignee: KLEMM+SOHN GMBH & CO. KG, Stuttgart (DE)

(21) Appl. No.: 13/065,573

(22) Filed: Mar. 24, 2011

(51) Int. Cl. A01H 5/00

(52) U.S. Cl. PLT/413

(57) ABSTRACT

A new Calibrachoa plant named ‘KLECA10217’ particularly distinguished by having double flowers, early flowering, continuously flowers during summer, and has double flower type stability during the hot season, is disclosed.

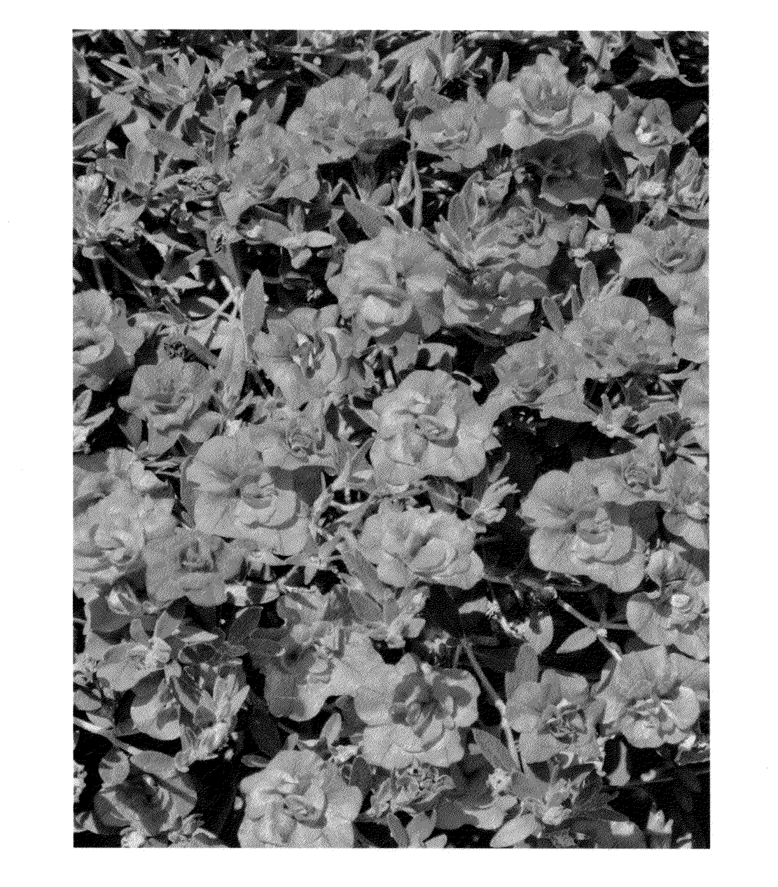

### GENUS AND SPECIES

Calibrachoa sp.

### VARIETY DENOMINATION

‘KLECA10217’

### BACKGROUND OF THE NEW PLANT

The present invention comprises a new and distinct variety of Calibrachoa, botanically known as Calibrachoa sp. and hereinafter referred to by the variety name ‘KLECA10217’. The new variety was the result of a controlled cross conducted in the Summer of 2007 in Stuttgart, Germany, between the female parent, proprietary Calibrachoa plant ‘CA 06 0149’, and the male parent, proprietary Calibrachoa plant ‘J 208’. A single plant selection was subsequently chosen for further evaluation and asexual propagation.

The new variety was first propagated in May 2008 in Stuttgart, Germany and has been asexually reproduced repeatedly by vegetative cuttings and in-vitro propagation over more than forty generations. The present invention has been found to retain its distinctive characteristics through successive asexual propagations via vegetative cuttings.

Plant Breeder's Rights for this variety were applied for in Canada on Mar. 19, 2010. ‘KLECA10217’ has not been made publicly available or sold more than one year prior to the filing date of this application.

### SUMMARY OF THE INVENTION

The following are the most outstanding and distinguishing characteristics of this new variety when grown under normal horticultural practices in a glass greenhouse in Stuttgart, Germany.

1. Double flowers;

2. Early flowering; and

3. Continuous summertime flowering and double flower stability during hot season.

### DESCRIPTION OF THE PHOTOGRAPH

This new Calibrachoa plant is illustrated by the accompanying photograph which shows inflorescences and foliage of the plant. The colors shown are as true as can be reasonably obtained by conventional photographic procedures. The photograph was taken in May 2010 of a 5-month old Calibrachoa plant grown in a 10 centimeter pot in a glass greenhouse in Stuttgart, Germany under normal horticultural practices.

### DESCRIPTION OF THE NEW VARIETY

The following detailed descriptions set forth the distinctive characteristics of ‘KLECA10217’. The data which defines these characteristics were collected from asexual reproductions carried out in Stuttgart, Germany. The plant history was taken in May 2010 of 5-month old plants grown in 10 centimeter pots in a glass greenhouse with a minimum pinch date of one week. Color readings were taken under natural light. Color references are primarily to the RHS Colour Chart of The Royal Horticultural Society of London (R.H.S.), Fifth Edition (2007).

### DETAILED BOTANICAL DESCRIPTION OF THE NEW PLANT

Classification:
   Family.—Solanaceae.
   Botanical.—Calibrachoa sp.
   Common.—Calibrachoa Hybrid.
Parentage:
   Female.—The proprietary Calibrachoa plant ‘CA 06 0149’.
   Male.—The proprietary Calibrachoa plant ‘J 208’.
Growth:
   Form.—Flat.
   Habit.—Trailing.
   Height (measured from the top of the soil).—4.0 cm.
   Width (horizontal plant diameter).—17.0 cm.
   Time to produce a finished flowering plant.—8 weeks after potting.
   Outdoor plant performance.—Continuously flowering, stable double flower.
   Time to initiate and develop roots.—14 days.
   Root description.—Moderate density, freely branching.
   Root color.—RHS 155D.
Stems:
   Length of basal branches.—9.0 cm.
   Internode length.—1.2 cm to 2.0 cm.
   Diameter of branches (from midpoint).—8.0 cm to 10.0 cm.
   Stem color.—RHS 149B.
   Texture.—Smooth.
   Anthocyanin.—Present. Color: RHS 166A.
   Pedicel.—Color: RHS 149C Length: 1.5 cm to 2.0 cm Diameter: 0.3 cm Texture: Smooth.
Leaves:
   Arrangement.—Alternate.
   Size.—Length: 2.4 cm to 3.5 cm Width: 0.7 cm to 1.3 cm.
   Shape.—Elliptic.
   Apex.—Broad acute.
   Base.—Attenuate.
   Margin.—Entire.
   Color.—Immature leaves: Upper surface: RHS 146A Lower surface: RHS 146B Mature leaves: Upper surface: RHS 137C Lower surface: RHS 137D.
   Texture (both surfaces).—Smooth.
   Venation pattern.—Pinnate.
   Venation color.—RHS 145A.
   Petioles.—Absent.
Flower bud:
   Shape.—Irregular to oblong.
   Size.—Length: 2.0 cm Diameter: 0.8 cm.
   Color at tight bud.—RHS 73D.
Inflorescence:
   Blooming habit.—Continuous flowering during summertime.
   Inflorescence type.—Double flower, determinate inflorescence.
   Floret type.—Double flowers.
   Lastingness of individual blooms on the plant.—6 days.
   Fragrance.—Absent.
Flowers:
   Immature flower.—Diameter: Horizontal: 2.5 cm Vertical: 1.9 cm Color: Upper surface: RHS 73A Lower surface: RHS 73B.
   Mature flower.—Diameter: Horizontal: 3.0 cm Vertical: 2.2 cm Color: Upper surface: RHS 73A Lower surface: RHS 73C.
   Corolla tube.—Color inside: RHS 73A and RHS 155D Color outside: RHS 33C Tube length: 1.0 cm Texture (both inside and outside): Smooth.
   Petals.—Apex: Obverse Base: Open and fused into the tube Margin: Entire Waviness of petals: Weak Lobation: Weak Texture (both surfaces): Smooth.
   Sepals.—Calyx (composition): Rosette, actinomorphic Shape: Lanceolate to elliptic Apex: Acute Base: Open and tube is fused into the base Margin: Entire Length: 1.2 cm Width: 0.3 cm Color: Upper surface: RHS 138B Lower surface: RHS 138C Texture (both surfaces): Smooth.
Reproductive organs:
   Stamens.—Absent.
   Pistils.—Number: 1 Length: 0.3 cm Diameter: 0.2 cm Stigma color: RHS 149A Style color: RHS 149A.
Fruit and seed set: No fruit or seed set observed.

### COMPARISON WITH PARENTAL AND COMMERCIAL VARIETIES

‘KLECA10217’ differs from the female parent Calibrachoa plant ‘CA 06 0149’, in that ‘KLECA10217’ has a flower diameter of 2.5 centimeters and good branching, whereas ‘CA 06 0149’ has a flower diameter of 1.3 centimeters and poor branching.

‘KLECA10217’ differs from the male parent Calibrachoa plant ‘J 208’ in that ‘KLECA10217’ has double flowers, whereas ‘J 208’ has single flowers.

‘KLECA10217’ differs from the commercial cultivar ‘MF Double Amethyst’, in that ‘KLECA10217’ has red-purple colored flowers, whereas, ‘MF Double Amethyst’ has violet-blue colored flowers.

We claim:

1. A new and distinct variety of Calibrachoa plant named ‘KLECA10217’ as shown and described herein.
